(19) United States
(12) Patent Application Publication
(10) Pub. No.: US 2015/0374832 A1
(43) Pub. Date: Dec. 31, 2015

(54) EPINEPHRINE FORMULATIONS FOR MEDICINAL PRODUCTS

(71) Applicant: YS PHARM TECH, San Diego, CA (US)

(72) Inventor: Yosyong Surakitbanharn, San Diego, CA (US)

(21) Appl. No.: 14/767,419

(22) PCT Filed: Feb. 12, 2014
(86) PCT No.: PCT/US14/16049

§ 371 Date: Aug. 12, 2015

Related U.S. Application Data
(60) Provisional application No. 61/763,843, filed on Feb. 12, 2013.

(51) Int. Cl. A61K 47/40; A61K 9/00; A61K 31/137

(52) U.S. Cl. CPC A61K 47/40; A61K 31/137; A61K 9/0019; A61K 9/0014; A61K 9/0043; A61K 9/0048; A61K 9/0073

(57) ABSTRACT

The invention relates to compositions of epinephrine formulation in an aqueous solution that enhances the chemical stability of epinephrine and consequently extends the product shelf life. The formulation comprises epinephrine or a salt thereof, a tonicity modifier, and a complexing agent, in an aqueous solution adjusted to a pH of about 2-7. A process for manufacturing and methods of using the formulation for the medicinal products are also provided.

### CROSS-REFERENCE TO RELATED APPLICATIONS

This application claims benefit of priority of U.S. Provisional Patent Application Ser. No. 61/763,843, filed Feb. 12, 2013, entitled “Chemical Stability Enhancement of Epinephrine in Aqueous Solution for Medicinal Products”, owned by the assignee of the present invention and herein incorporated by reference in its entirety.

### FIELD OF THE INVENTION

The invention generally relates to the chemical stability enhancement of epinephrine in aqueous solution of medicinal products used for treatments of various diseases, e.g. anaphylactic shock, cardiac arrest, bronchial asthma and glaucoma.

### BACKGROUND OF THE INVENTION

Epinephrine, more commonly known as adrenaline, is a hormone secreted by the medulla of the adrenal glands. Strong emotions such as fear or anger cause epinephrine to be released into the bloodstream, which causes an increase in heart rate, muscle strength, blood pressure, and sugar metabolism. This reaction, known as the “Flight or Fight Response”, prepares the body for strenuous activity. Epinephrine is found in small amounts in the body and is essential for maintaining cardiovascular homeostasis because of its ability to divert blood to tissues under stress.

In medicine, epinephrine is used mainly as a stimulant in cardiac arrest, as a vasoconstrictor in shock, and as a bronchodilator and antispasmodic in bronchial asthma. Its uses also include at least combating low blood pressure during hemorrhagic, allergic or anaphylactic shock; opening the airways during thematic attack; restricting the distribution of locally administered drugs such as local anesthetics; reducing nasal congestion; reducing the amount of fluid in the eye to decrease intraocular pressure and/or as a performance aid in emergency situations.

Allergic emergencies, such as anaphylaxis, are a growing concern, given the increasing awareness of members of the public of their frequency and potential severity. Anaphylaxis is a sudden, severe, systemic allergic reaction that can be fatal, in many cases, if left untreated. Anaphylaxis can involve various areas of the body, such as the skin, respiratory tract, gastrointestinal tract, and cardiovascular system. Acute symptoms occur from within minutes to two hours after contact with the allergy-causing substance, but in rare instances onset may be delayed by as much as four hours. Contact with anaphylaxis-inducing agents, and the severity of the resulting anaphylactic reaction, can be extremely unpredictable. Accordingly, allergists recommend that persons who have a personal or family history of anaphylaxis be prepared to self-administer emergency treatment at all times. Additionally, adults charged with caring for children who are at risk for anaphylaxis should also be prepared to administer anti-anaphylactic first aid.

The symptoms of anaphylaxis include one or more of the following, generally within 1 to about 15 minutes of exposure to the antigen: agitation, a feeling of uneasiness, flushing, palpitations, paresthesias, pruritus, throbbing in the ears, coughing, sneezing, urticaria, angioedema, difficulty breathing due to laryngeal edema or brochospasm, nausea, vomiting, abdominal pain, diarrhea, shock, convulsions, incontinence, unresponsiveness and death. An anaphylactic reaction may include cardiovascular collapse, even in the absence of respiratory symptoms.

Due to its vasoconstrictive effects, epinephrine is the drug of choice for treating anaphylaxis. Allergy patients undergoing immunotherapy may receive an adrenaline rinse before the allergen extract is administered, thus reducing the immune response to the administered allergen. Because of various expressions of α1 or β2 receptors, depending on the patient, administration of epinephrine may raise or lower blood pressure, depending on whether or not the net increase or decrease in peripheral resistance can balance the positive inotropic and chronotropic effects of adrenaline on the heart, effects that increase the contractility and rate, respectively, of the heart.

Epinephrine is a sympathomimetic catecholamine Chemically, epinephrine is (−)-3,4-Dihydroxy-α-[(methylamino)methyl]benzyl alcohol. Epinephrine in aqueous solution deteriorates rapidly on exposure to air or light or heat and discolors to pink from the oxidation to adrenochrome and to brown from the formation of melanin.

Epinephrine is a catechol compound that is sensitive to oxidation to o-quinones, which can react further to form highly colored compounds. Epinephrine can thus react to form adrenochrome, a highly colored indole derivative. The rate of this reaction increases with pH, temperature and by the presence of metal ions, such as aluminum from various rubbers and iron from amber glassware. Epinephrine solutions may also lose potency as a result of racemization, and protection from light minimizes this form of instability.

The modification or degradation of the catechol amines is undesirable for a number of reasons. Modification of the catechol amine results in loss of titer of the active ingredient, formation of compounds which may have undesirable physiological effects, and the appearance of a dark color, which makes the solution offensive and unmarketable. The initial loss of active compound due to auto-oxidation during the preparation and packaging of such a solution is substantial despite the fact that such procedures are carried out as nearly as practically possible in an inert atmosphere. Such a solution must be stored under refrigeration in order to decrease the rate of deterioration of the compound and thus prolong its shelf-life.

It is a standard practice, in order to stabilize adrenergic compounds such as catechol amines against auto-oxidation, to combine the same with an antioxidant. Various antioxidants which have been used to stabilize catechol amine solution in a variety of formulations such as aerosols, eye-drops, injections etc. including metabisulfite, bisulfate, sulfite, ascorbic acid, thiglycollate, thioglycerol, cysteine, propyl gallate and formaldehyde sulfoxylate (References: GB 425678, GB 930452, U.S. Pat. No. 3,149,035, U.S. Pat. No. 3,966,905, CA 981182, US 2008/0269347 A1, DD-A1-150 694, WO 94/13274, WO 97/16196, WO98/20869, U.S. Pat. No. 4,734,438).

For anaphylactic treatment, the usual epinephrine concentration is 0.3-0.5 mg in 1:1000 dilution for subcutaneous or intramuscular injection, which is commercially available in auto injector devices such as Epipen®, Twinject® and Auvi-Q™. Epipen®, according to its prescribing information, is designed to deliver a minimum of 0.3 mg epinephrine in a 0.3 mL injection volume. Its composition in 1 mL water for injection consists of either 1.0 mg epinephrine as free base, 6.0 mg sodium chloride, 1.7 mg sodium metabisulfite and hydrochloric acid to adjust pH 2.2-5.0. Twinject® has a comparable composition to Epipen®, but uses sodium bisulfite instead of sodium metabisulfite and includes chlorobutanol as a preservative. Auvi-Q™ has a comparable composition to Twinject® and an absence of chlorobutanol.

Note that sodium metabisulfite or sodium bisulfite, which is commonly used in the conventional epinephrine formulations as an antioxidant, has been associated with some other severe allergic reactions. In addition, sodium bisulfite can directly react with epinephrine to produce a degradation product, epinephrine sulfonic acid (ESA). The increase of ESA in the epinephrine formulation containing sodium metabisulfite or sodium bisulfite could be greater than 15% at the end of product life. The safety and/or toxicity of ESA in commercial epinephrine products for anaphylactic treatment are still not well understood. In addition, the potency of epinephrine also could be substantially degraded to nearly 10% due to such reaction at the end of product life.

This Background is provided to introduce a brief context for the Summary and Detailed Description that follow. This Background is not intended to be an aid in determining the scope of the claimed subject matter nor be viewed as limiting the claimed subject matter to implementations that solve any or all of the disadvantages or problems presented above.

### SUMMARY OF THE INVENTION

The present invention provides the compositions and methods of using a novel formulation to enhance the chemical stability of epinephrine, e.g., in aqueous solutions. The formulation of epinephrine utilizes a complexing agent, which is a native or modified cyclodextrin derivative to provide an inclusion complex with epinephrine. The invention is based on findings of improved stability of epinephrine in the presence of a complexing agent in an aqueous solution against thermal and/or oxidative degradations.

In one embodiment, the formulation comprises epinephrine or its salts, a tonicity modifier and a complexing agent in an aqueous solution. The epinephrine or its salts is selected from the group consisting of epinephrine, epinephrine bitartrate, and epinephrine hydrochloride. The epinephrine or its salts as free base equivalent is in the range from 0.0001% to 5% depending on the therapeutic treatments. For example, epinephrine is in the range of 0.0001-1% for injectable formulations; 0.01-1% for topical formulations and nasal formulations; 0.1-2% for ophthalmic formulations and ophthalmic drops; and 1-5% for inhalation formulations. The tonicity modifier is selected from the group consisting of sodium chloride and dextrose and a combination thereof. The tonicity modifier is used to adjust the solution osmolality close to the physiological osmolality in the range of about 200-400 mOsm/kg. The complexing agent is selected from the group consisting of native and modified cyclodextrin derivatives including α-cyclodextrin, β-cyclodextrin, γ-cyclodextrin, modified α-cyclodexin, modified β-cyclodextin and modified γ-cyclodextrin and a combination thereof, preferably modified β-cyclodextin, i.e. hydroxypropyl β-cyclodextrin, sulfobutyl ether β-cyclodextrin, and randomly methylated β-cyclodextrin, etc. The molar ratio of cyclodextrin to epinephrine is used in the range from 0.01:1 to 10:1. The aqueous based media, preferably water for injection, is adjusted to a pH of about 2-7 using hydrochloric acid (HCl) and/or sodium hydroxide (NaOH).

In another embodiment, the formulation further contains a chelating agent. The chelating agent is selected from the group consisting of edetic acid (etylenediaminetetraacetic acid) and its salts including edetate calcium disodium, edetate disodium, edetate disodium anhydrous, edetae sodium and a combination thereof, in the range from 0.001% to 2%.

In another embodiment, the formulation optionally contains an antioxidant. The antioxidant is selected from the group consisting of oxine, boric acid, borate ascorbic acid, erythorbic acid, malic acid, acetylcysteine, thioglycerol cysteine, citric acid, polyvinylpyrrolidone, and a combination thereof.

In another embodiment, the formulation can be used in conjunction with an administrative device. The device is selected from a group consisting of: a pre-filled syringe for use in a manual and/or auto injector, a prefilled syringe for use in delivering a solution spray or droplet, an actuator for use in delivering a solution spray, a nebulizer for use in delivering a solution aerosol; and an applicator for ophthalmic administration and for topical administration.

In another embodiment, the invention provides a method for treating diseases such as anaphylactic shock, cardiac arrest, bronchial asthma, restricting the distribution of locally administered drugs with local anesthetics for both intact and broken skins; reducing nasal congestion, and reducing the amount of fluid in the eye to decrease intraocular pressure and treating glaucoma to a subject, by administering an effective amount of the formulation in the present invention.

In another embodiment, the formulation of the present invention can be administered by intramuscular injection, subcutaneous injection, intravenous injection, ocular injection, ophthalmic formulation and ophthalmic drop, buccal injection, buccal spray, sublingual spray, nasal spray and nasal drop, inhalation, and topical application. This Summary is provided to introduce a selection of concepts in a simplified form. The concepts are further described in the Detailed Description section. Elements or steps other than those described in this Summary are possible, and no element or step is necessarily required. This Summary is not intended to identify key features or essential features of the claimed subject matter, nor is it intended for use as an aid in determining the scope of the claimed subject matter. The claimed subject matter is not limited to implementations that solve any or all disadvantages noted in any part of this disclosure.

### BRIEF DESCRIPTION OF THE DRAWINGS

FIG. 1 illustrates the UV absorption of epinephrine as a function of sulfobutyl ether β-cyclodextrin (Captisol®) concentration in an aqueous solution (pH 3.5) at room temperature.

FIG. 2 illustrates a graphical method for determining the equilibrium constant between epinephrine and sulfobutyl ether β-cyclodextrin (Captisol®) in an aqueous solution (pH 3.5) at room temperature.

### DETAILED DESCRIPTION OF THE INVENTION

In order to prevent the thermal and/or oxidative degradations of epinephrine, the commercial formulations contain a conventional antioxidant of sulfite or bisulfite related compounds such as sodium bisulfite and/or sodium metabisulfite. The products include examples such as epinephrine injections for anaphylactic treatment, i.e., Epipen®, Twinject® and Auvi-Q™ (epinephrine auto injectors). These antioxidants can directly react with epinephrine, resulting in substantial degradation of epinephrine potency and generating a degradation product, epinephrine sulfonic acid (ESA), which increases with time and becomes a major limiting factor to the product shelf life.

The sulfite or bisulfite related compounds in foods and/or medications could cause severe allergy or asthma reactions. For instance, some people have experienced severe reactions from sulfite-containing medications including intravenous drugs and inhaled medications, these reactions including flushing, hives, and a drop in lung function. The present invention provides the compositions of a “sulfite or bisulfite free” formulation of epinephrine, which significantly improves the chemical stability and eliminates the patient's risk of an exposure to severe allergy or asthma reaction from the aforementioned antioxidant.

The present invention provides compositions and methods of using a novel formulation to enhance the chemical stability of epinephrine in aqueous solution and to consequently extend the product shelf life. The invention also provides a safer medication for patients by eliminating a need for a conventional antioxidant, e.g., sulfite or bisulfite related compounds in the formulation, that degrades the epinephrine potency, generates a degradation product (epinephrine sulfonic acid, ESA), and potentially causes the subsequent severe asthma and/or allergy reactions. Therefore, the present invention would reduce the patient's risks of exposures to high ESA levels and unnecessary asthma and/or allergy reactions as currently found in the commercial products.

The pharmaceutical formulation comprises epinephrine or salts thereof, a complexing agent, a tonicity modifier, and a chelating agent, in an aqueous pH solution.

The present invention uses epinephrine or its salts as an active pharmaceutical ingredient selected from epinephrine, epinephrine bitartrate, and epinephrine hydrochloride, preferably epinephrine. Epinephrine or its salts as free base equivalent is used within the range of about 0.0001-5% depending on the therapeutic treatments. For example, 0.0001-0.01% injection is used for a vasoconstrictor to prolong local anesthetic effects; 0.01-1% injection is used for allergy, anaphylaxis, cardiac arrest, bronchodilation and hypersensitivity reaction; 0.01-1% topical formulation is used as a vasoconstrictive agent in combination with other active ingredients for anesthetic pretreatment of local analgesia on broken skin; 0.1-2% ophthalmic drops for glaucoma, and 0.1-5% for nasal congestion and for inhalation for asthma attack.

The present invention provides water for injection (WFI) as a diluent. The pH of WFI is adjusted using hydrochloric acid and/or sodium hydroxide. The pH is adjusted to enhance the epinephrine stability and control the equilibrium association constant between epinephrine and cyclodextrin within a range of about 2-7.

The present invention provides sodium chloride and/or dextrose and a combination thereof, preferably sodium chloride, as a tonicity modifier to adjust the solution osmolality within a range of about 200-400 mOsm/kg.

The present invention provides cyclodextrin as a complexing agent to chemically form an inclusion complex with epinephrine. The complexing agent is selected from the group consisting of native and/or modified cyclodextrin derivatives including α-cyclodextrin, β-cyclodextrin, γ-cyclodextrin, modified α-cyclodexin, modified β-cyclodextin, and modified γ-cyclodextrin, and a combination thereof, preferably the modified β-cyclodextin, i.e., hydroxypropyl β-cyclodextrin (Kleptose® HPB, Kleptose® HP, Trappsol® HPB), sulfobutyl ether β-cyclodextrin (Captisol®), and randomly methylated β-cyclodextrin (Kleptose® Crysmeb Exp) etc.

Cyclodextrins (sometimes called cycloamyloses) are a family of compounds made up of sugar molecules bound together in a ring (cyclic oligosaccharides). For example, sulfobutyl ether β-cyclodextrin is a polyanionic beta-cyclodextrin derivative with a sodium sulfonate salt separated from the lipophilic cavity by a butyl ether spacer group, or sulfobutyl ether (SBE). Sulfobutyl ether β-cyclodextrin is not a single chemical species, but comprised of a multitude of polymeric structures of varying degrees of substitution and positional/regional isomers. Sulfobutyl ether β-cyclodextrin is an approved pharmaceutical ingredient for commercial injectable products. Hydroxypropyl β-cyclodextrin is the most widely used modified cyclodextrin, with the lipophilic cavity formed by 7 glucose units. It has the most extensive collection of safety data in the technical literature with no adverse reactions reported, and is approved for use for injectable products and parenteral products. The molar ratio of cyclodextrin to epinephrine is within a range of about 0.1:1-10:1.

The present invention also includes a chelating agent to prevent epinephrine degradation in a presence of trace metallic catalyst. The chelating agent is selected from the group consisting of edetic acid or its salts including edetate calcium disodium, edetate disodium, edetate disodium, anhydrous, edetate sodium, and a combination thereof, within a range about 0.01-2%.

The present invention optionally contains an antioxidant. The antioxidant is selected from oxine, boric acid, borate, ascorbic acid, erythorbic acid, malic acid acetylcysteine, thioglycerol cysteine, citric acid, polyvinylpyrrolidone and a combination thereof.

The present invention can be used with various administrative devices. The device is selected from a group consisting of a pre-filled syringe for use in manual and/or auto injectors, a prefilled syringe or other packaging configurations for use in delivering solution sprays or droplets, actuators for use in delivering solution sprays, and nebulizers for use in delivering solution aerosols.

The present invention provides a method for treating anaphylactic shock, cardiac arrest, bronchial asthma and glaucoma; restricting the distribution of locally administered drugs such as local anesthetics; reducing nasal congestion, and reducing the amount of fluid in the eye to decrease intraocular pressure to a subject by administering an effective amount of the formulation to a subject by intramuscular injection, subcutaneous injection, intravenous injection, buccal injection, buccal absorption, nasal spray inhalation, sublingual absorption, intraocular absorption and topical absorption.

The following examples are provided to further explain the invention.

### EXAMPLES

### Example 1

The compositions and method for preparation of various formulations containing sulfobutyl ether β-cyclodextrin (SBE-CD)

Formulation 1:

| Ingredient | Concentration (mg/mL) |
|---|---|
| Epinephrine (as free base) | 1.0 |
| Sulfobutyl ether β-cyclodextrin | 6.0 |
| Sodium Chloride | 8.5 |
| HCl and/or NaOH (adjust to target pH 3.5) | — |
| Water for Injection (WFI, adjust to volume) | q.s. |

Formulation 2:

| Ingredient | Concentration (mg/mL) |
|---|---|
| Epinephrine (as free base) | 1.0 |
| Sulfobutyl ether β-cyclodextrin | 12.0 |
| Sodium Chloride | 8.5 |
| HCl and/or NaOH (adjust to target pH 3.5) | — |
| Water for Injection (WFI, adjust to volume) | q.s. |

Formulation 3:

| Ingredient | Concentration (mg/mL) |
|---|---|
| Epinephrine (as free base) | 1.0 |
| Sulfobutyl ether β-cyclodextrin | 24.0 |
| Sodium Chloride | 8.5 |
| HCl and/or NaOH (adjust target pH 3.5) | — |
| Water for Injection (WFI, adjust to volume) | q.s. |

Formulation 4:

| Ingredient | Concentration (mg/mL) |
|---|---|
| Epinephrine (as free base) | 1.0 |
| Sulfobutyl ether β-cyclodextrin | 60.0 |
| Sodium Chloride | 8.5 |
| HCl and/or NaOH (adjust to target pH 3.5) | — |
| Water for Injection (WFI, adjust to volume) | q.s. |

Formulation 5:

| Ingredient | Concentration (mg/mL) |
|---|---|
| Epinephrine (as free base) | 1.0 |
| Sulfobutyl ether β-cyclodextrin | 6.0 |
| Sodium Chloride | 8.5 |
| HCl and/or NaOH (adjust to pH 5.5) | — |
| Water for Injection (WFI, adjust to volume) | q.s. |

Formulation 6:

| Ingredient | Concentration (mg/mL) |
|---|---|
| Epinephrine (as free base) | 1.0 |
| Sulfobutyl ether β-cyclodextrin | 12.0 |
| Sodium Chloride | 8.5 |
| HCl and/or NaOH (adjust to pH 5.5) | — |
| Water for Injection (WFI, adjust to volume) | q.s. |

Formulation 7:

| Ingredient | Concentration (mg/mL) |
|---|---|
| Epinephrine (as free base) | 1.0 |
| Sulfobutyl ether β-cyclodextrin | 24.0 |
| Sodium Chloride | 8.5 |
| HCl and/or NaOH (adjust pH 5.5) | — |
| Water for Injection (WFI, adjust to volume) | q.s. |

Formulation 8:

| Ingredient | Concentration (mg/mL) |
|---|---|
| Epinephrine (as free base) | 1.0 |
| Sulfobutyl ether β-cyclodextrin | 60.0 |
| Sodium Chloride | 8.5 |
| HCl and/or NaOH (adjust to pH 5.5) | — |
| Water for Injection (WFI, adjust to volume) | q.s. |

A method for preparation of Formulations 1 through 8 is described as follows:
a) Sparge WFI with nitrogen gas to remove dissolved oxygen
b) Add and completely dissolve sodium chloride
c) Add and completely dissolve sulfobutyl ether 3-cyclodextrin
d) Adjust pH to 2.0 using HCl and/or NaOH solutions
e) Add and completely dissolve epinephrine
f) Adjust pH to a target pH at 3.5 (Formulations 1-4) or 5.5 (Formulations 5-8) using HCl and/or NaOH solutions
g) Adjust the batch size to volume using adjusted pH WFI.

### Example 2

The compositions and method for preparation of various formulations containing hydroxypropyl β-cyclodextrin (HP-CD) are shown as follows:

Formulation 9:

| Ingredient | Concentration (mg/mL) |
|---|---|
| Epinephrine (as free base) | 1.0 |
| Hydroxypropyl β-cyclodextrin | 4.0 |
| Sodium Chloride | 8.5 |
| HCl and/or NaOH (adjust to target pH 3.5) | — |
| Water for Injection (WFI, adjust to volume) | q.s. |

Formulation 10:

| Ingredient | Concentration (mg/mL) |
|---|---|
| Epinephrine (as free base) | 1.0 |
| Hydroxypropyl β-cyclodextrin | 8.0 |
| Sodium Chloride | 8.5 |
| HCl and/or NaOH (adjust to target pH 3.5) | — |
| Water for Injection (WFI, adjust to volume) | q.s. |

Formulation 11:

| Ingredient | Concentration (mg/mL) |
|---|---|
| Epinephrine (as free base) | 1.0 |
| Hydroxypropyl β-cyclodextrin | 16 |
| Sodium Chloride | 8.5 |
| HCl and/or NaOH (adjust target pH 3.5) | — |
| Water for Injection (WFI, adjust to volume) | q.s. |

Formulation 12:

| Ingredient | Concentration (mg/mL) |
|---|---|
| Epinephrine (as free base) | 1.0 |
| Hydroxypropyl β-cyclodextrin | 40.0 |
| Sodium Chloride | 8.5 |
| HCl and/or NaOH (adjust to target pH 3.5) | — |
| Water for Injection (WFI, adjust to volume) | q.s. |

Formulation 13:

| Ingredient | Concentration (mg/mL) |
|---|---|
| Epinephrine (as free base) | 1.0 |
| Hydroxypropyl β-cyclodextrin | 4.0 |
| Sodium Chloride | 8.5 |
| HCl and/or NaOH (adjust to pH 5.5) | — |
| Water for Injection (WFI, adjust to volume) | q.s. |

Formulation 14:

| Ingredient | Concentration (mg/mL) |
|---|---|
| Epinephrine (as free base) | 1.0 |
| Hydroxypropyl β-cyclodextrin | 8.0 |
| Sodium Chloride | 8.5 |
| HCl and/or NaOH (adjust to pH 5.5) | — |
| Water for Injection (WFI, adjust to volume) | q.s. |

Formulation 15:

| Ingredient | Concentration (mg/mL) |
|---|---|
| Epinephrine (as free base) | 1.0 |
| Hydroxypropyl β-cyclodextrin | 16.0 |
| Sodium Chloride | 8.5 |
| HCl and/or NaOH (adjust pH 5.5) | — |
| Water for Injection (WFI, adjust to volume) | q.s. |

Formulation 16:

| Ingredient | Concentration (mg/mL) |
|---|---|
| Epinephrine (as free base) | 1.0 |
| Hydroxypropyl β-cyclodextrin | 40.0 |
| Sodium Chloride | 8.5 |
| HCl and/or NaOH (adjust to pH 5.5) | — |
| Water for Injection (WFI, adjust to volume) | q.s. |

Method for preparation of Formulations 9 through 16 is described as follows:
a. Sparge WFI with nitrogen gas to remove dissolved oxygen
b. Add and completely dissolve sodium chloride
c. Add and completely dissolve hydroxypropyl 3-cyclodextrin
d. Adjust pH to 2.0 using HCl and/or NaOH solutions
e. Add and completely dissolve epinephrine
f. Adjust pH to a target pH at 3.5 (Formulations 9-12) or 5.5 (Formulations 13-16) using HCl and/or NaOH solutions
g. Adjust the batch size to volume using adjusted pH WFI.

Example 1 provides the compositions of Formulations 1-8 and methods of manufacturing 0.1% (1.0 mg/mL) epinephrine formulations at two pH levels (3.5 & 5.5) containing 0.6-6.0% (6.0-60.0 mg/mL) of sulfobutyl ether β-cyclodextrin (SBE-CD) in a presence of 0.85% (8.5 mg/mL) sodium chloride. The formulations were tested side-by-side with a commercial formulation containing 0.1% (1.0 mg/mL) epinephrine, 0.15% (1.5 mg/mL) sodium bisulfite and 0.85% (8.5 mg/mL) sodium chloride at pH 3.5 under an accelerated stability condition at 50° C. for six weeks.

The epinephrine potency from the commercial formulation decreased with time approximately 10% and 40% from its initial value after 2 and 6 weeks, respectively; whereas those formulations provided by the present invention were much more stable and varied approximately not more than 5% of their initial values after six weeks. A degradation product, epinephrine sulfonic acid (ESA) in the commercial formulation, was found approximately 17-20% and 50% after 2 and 6 weeks, respectively, in the commercial formulation; whereas those from the present invention formulations were detected at a level less than 0.1% after 6 weeks. Another degradation product, e.g., adrenalone, was detected at a level less than 0.1% in the present invention formulations after 6 weeks.

Example 2 provides the compositions of Formulation 9-16 and methods of manufacturing 0.1% (1.0 mg/mL) epinephrine formulations at two pH levels (3.5 & 5.5) containing 0.4-4.0% (4.0-40.0 mg/mL) hydroxypropyl 3-cyclodextrin (HP-CD) in a presence of 0.85% (8.5 mg/mL) sodium chloride. The epinephrine formulations of the present invention were found to be fairly stable. For example, epinephrine potencies of the formulations at pH 3.5 varied approximately not more than 5% of their initial values after six weeks at 50° C.

As described above, the examples of epinephrine stabilization from all the formulations of the present invention in both Examples 1 and 2 were significantly improved against the thermal and/or oxidative degradations compared to the conventional commercial formulation.

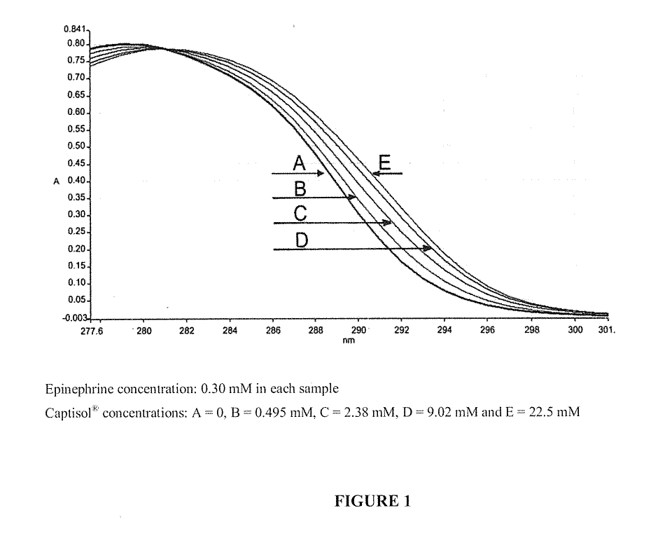

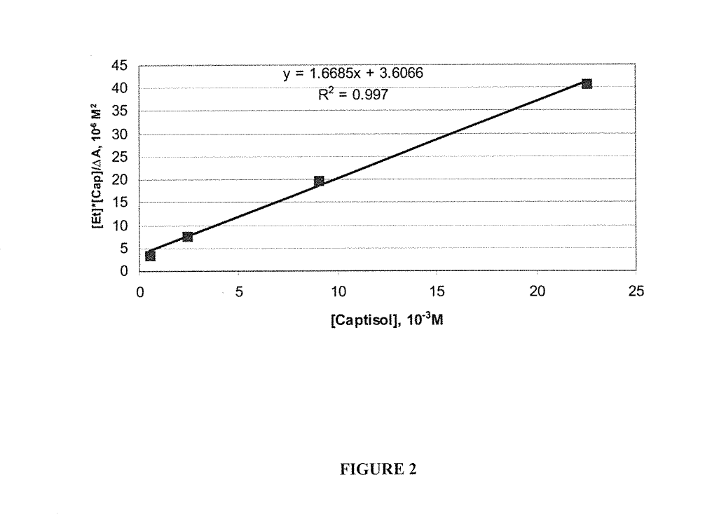

In addition, an equilibrium constant of inclusion complex between epinephrine and sulfobutyl ether β-cyclodextrin (SBE-CD) in aqueous solution was determined using a spectroscopy technique, i.e., UV spectrophotometer as shown in FIG. 1. The equilibrium constant (K) between epinephrine and SBE-CD was determined by increasing the molar concentration ratio of SBD-CD to epinephrine in solution and measuring the spectral shift (ΔA) as shown FIG. 2 at pH 3.5. The K value was derived from the slope and intercept of a linear relationship from a plot between [Epi][SBD-CD]/ΔA and [SBD-CD] according to the Hildebrand and Benesi equation as shown in FIG. 2 (Rong Liu, Editor, Water-Insoluble Drug Formulation, Interpharm Press, 2000; and F. Cramer et. al., The Formation of Inclusion Compounds of α-Cyclodextrin in Aqueous Solutions, J. Am. Chem. Soc., 89, 14-20, 1967, the disclosures of which are incorporated herein by reference in their entireties). The K values were determined and found to be pH dependent, for example: 488 M⁻¹ at pH 3.5 (FIG. 2) and 111 M⁻¹ at pH 5.5.

The formulations provided by the present invention contain epinephrine or a salt thereof, a tonicity modifier and a complexing agent in an aqueous based media. The epinephrine or its salts, is selected from epinephrine, epinephrine bitartrate, and epinephrine hydrochloride. The epinephrine or its salts as free base equivalent is in the range from 0.0001-5% depending on the therapeutic treatments. The tonicity modifier is selected from sodium chloride, dextrose, and a combination thereof. The solution osmolality is adjusted to about 200-400 mOsm/kg. The complexing agent is selected from the group consisting of native and/or modified cyclodextrin derivatives including α-cyclodextrin, β-cyclodextrin, γ-cyclodextrin, modified α-cyclodexin, modified β-cyclodextin and modified γ-cyclodextrin and a combination thereof, preferably the modified β-cyclodextin, i.e., hydroxypropyl β-cyclodextrin (HP-CD) and sulfobutyl ether β-cyclodextrin (SBE-CD). The molar ratio of cyclodextrin to epinephrine is in the range from about 0.01:1 to 10:1. The aqueous based media, preferably water for injection, is adjusted using hydrochloric acid and/or sodium hydroxide to a pH range of about 2-7. The formulation further contains a chelating agent. The chelating agent is selected from the group consisting of edetic acid or its salts including edetate calcium disodium, edetate disodium, edetate disodium anhydrous, edetate sodium, and a combination thereof, in the range of from about 0.001% to 2%. The formulation optionally contains an antioxidant. The antioxidant is selected from oxine, boric acid, borate, ascorbic acid, erythorbic acid, malic acid acetylcysteine, thioglycerol cysteine, citric acid, polyvinylpyrrolidone, and a combination thereof.

The formulation of the present invention can be used with an administrative device. The device is selected from a group consisting of a pre-filled syringe for use in manual and/or auto injectors, a prefilled syringe for use in delivering solution sprays or droplets, actuators for use in delivering solution sprays, nebulizers for use in delivering solution aerosols from a nebulizer, ophthalmic drop containers for intraocular administration, and so on.

Details of non-injectable formulations are now described.

Topical Formulation:

Topical anesthetics have provided physicians with multiple options in anesthetizing open and intact skin. Epinephrine, in combination with other anesthetic agents such as lidocaine, tetracaine, and cocaine, has been used for analgesia of lacerations to the face and scalp. The topical formulation can be prepared in an aqueous based media as a liquid or gel formulation. It can be applied directly to the wound with an applicator. In one implementation, the present invention may provide enhanced stability of epinephrine formulation by incorporating cyclodextrin as complexing agent. The concentration of epinephrine may be used in the range of 0.01-1%.

Ophthalmic Formulation:

Ophthalmic epinephrine is used to treat certain types of glaucoma and to reduce the amount of fluid in the eye to decrease intraocular pressure. It may also be used in eye surgery. The formulation can be applied as an eye drop using an applicator or by ocular injection. The ophthalmic formulation of epinephrine can be significantly improved by using a cyclodextrin formulation according to the present invention. The concentration of epinephrine may be used in the range of 0.1-2%.

Nasal Formulation:

Nasal epinephrine has been shown to be effective for treating anaphylaxis and nasal congestion. Clinical studies have demonstrated that a needle-free nasal epinephrine formulation can succeed in achieving rapid absorption in peripheral blood comparable to EpiPen®. The nasal formulation can be prepared in a solution and administered via an actuator from the nasal spray bottle or via an applicator for nasal drop. The shelf life can be extended by the cyclodextrin formulation provided by the present invention. The concentration of epinepharine may be used in the rage of 0.01-1%

Inhalation Formulation:

Inhaled epinephrine has been widely used to manage the upper airway obstruction and croup. It is also recommended to use as a first-line treatment for bronchiolitis. The concentration of epinephrine may be used in the range of 1-5%.

The formulation of the present invention provides a method for treating anaphylactic shock, cardiac arrest, bronchial asthma and glaucoma; restricting the distribution of locally administered topical drugs such as local anesthetics for both intact and broken skin; reducing nasal congestion, and reducing the amount of fluid in the eye to decrease intraocular pressure. The method is performed by administering an effective amount of the formulation to a subject by intramuscular injection, subcutaneous injection, intravenous injection, ocular injection, ophthalmic formulation and ophthalmic drop, buccal injection, buccal spray, sublingual spray, nasal spray and nasal drop, inhalation, topical application.

1.-18. (canceled)

19. A pharmaceutical formulation comprising epinephrine or its salts, a complexing agent, and a tonicity modifier in an aqueous solution.

20. The pharmaceutical formulation as in claim 19, further comprising a chelating agent.

21. The pharmaceutical formulation as in claim 19, wherein the epinephrine or its salts, is selected from a group consisting of epinephrine, epinephrine bitartrate, and epinephrine hydrochloride.

22. The pharmaceutical formulation as in claim 19, wherein the epinephrine or its salts as free base equivalent is in the range from 0.0001% to 5%.

23. The pharmaceutical formulation as in claim 19, wherein the epinephrine or its salts as free base equivalent is in the range 0.0001-1.0% for injectable formulations.

24. The pharmaceutical formulation as in claim 19, wherein the epinephrine or its salts as free base equivalent is in the range 0.01-1.0% for topical and nasal spray formulations.

25. The pharmaceutical formulation as in claim 19, wherein the epinephrine or its salts as free base equivalent is in the range 0.1-2% for ophthalmic formulations.

26. The pharmaceutical formulation as in claim 19, wherein the epinephrine or its salts as free base equivalent is in the range 1-5% for inhalation formulations.

27. The pharmaceutical formulation as in claim 19, wherein the complexing agent is cyclodextrin selected from a group consisting of α-cyclodextrin, β-cyclodextrin, γ-cyclodextrin, modified α-cyclodexin, modified β-cyclodextin and modified γ-cyclodextrin, and combinations thereof.

28. The pharmaceutical formulation as in claim 19, wherein the complexing agent is a modified β-cyclodextin, namely sulfobutyl ether β-cyclodextrin.

29. The pharmaceutical formulation as in claim 19, wherein the complexing agent is a modified β-cyclodextin, namely hydroxyl propyl β-cyclodextrin.

30. The pharmaceutical formulation as in claim 19, wherein the molar ratio of cyclodextrin to epinephrine is in the range from 0.01:1 to 10:1.

31. The pharmaceutical formulation as in claim 20, wherein the chelating agent is selected from the group consisting of edetic acid and its salts including edetate calcium disodium, edetate disodium, edetate disodium anhydrous, edetate sodium, and combinations thereof.

32. The pharmaceutical formulation as in claim 19, further comprising an antioxidant.

33. The pharmaceutical formulation as in claim 32, wherein the antioxidant is selected from the group consisting of oxine, boric acid, borate ascorbic acid, erythorbic acid, malic acid, acetylcysteine, thioglycerol, cysteine, citric acid, polyvinylpyrrolidone and combinations thereof.

34. A method for treating anaphylactic shock, cardiac arrest, and bronchial asthma in a subject, said method comprising administrating to a subject in need thereof an effective amount of the formulation according to claim 19.

35. A method for treating glaucoma, said method comprising administering to a subject in need thereof an effective amount of the formulation according to claim 19.

36. A method for restricting the distribution of locally administered drugs for both intact and broken skins, said method comprising administering to a subject in need thereof an effective amount of the formulation according to claim 19.

37. A method for reducing nasal congestion in a subject, said method comprising administering to a subject in need thereof an effective amount of the formulation according to claim 19.

38. A method for reducing the amount of fluid in the eye to decrease intraocular pressure, said method comprising administering to a subject in need thereof an effective amount of the formulation according to claim 19.
